(19) United States
(12) Patent Application Publication
(10) Pub. No.: US 2007/0010551 A1
(43) Pub. Date: Jan. 11, 2007

(54) Quinoline derivatives as neutrophil elastase inhibitors and their use

(76) Inventors: Hakan Bladh, Lund (SE); Joakim Larsson, Lund (SE)

Correspondence Address: FISH & RICHARDSON P.C., P.O BOX 1022, MINNEAPOLIS, MN, 55440-1022, US

(21) Appl. No.: 10/569,571

(22) PCT Filed: Aug. 25, 2004
(86) PCT No.: PCT/SE04/01226

§ 371 Date: Feb. 24, 2006

(30) Foreign Application Priority Data
Aug. 28, 2003 (SE) 0302323-1

(51) Int. Cl. A61K 31/4704; C07D 215/233

(52) U.S. Cl. 514/312; 546/156

(57) ABSTRACT

There are provided novel compounds of formula (I) wherein R¹, R³, R⁴, R⁵, G¹, G², L and n are as defined in the Specification and optical isomers, racemates and tautomers thereof, and pharmaceutically acceptable salts thereof; together with processes for their preparation, compositions containing them and their use in therapy. The compounds are inhibitors of neutrophil elastase.

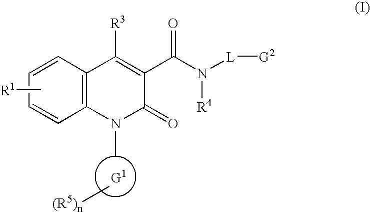

### FIELD OF THE INVENTION

This invention relates to novel quinoline derivatives, processes for their preparation, pharmaceutical compositions comprising them, and their use in therapy.

### BACKGROUND OF THE INVENTION

Elastases are possibly the most destructive enzymes in the body, having the ability to degrade virtually all connective tissue components. The uncontrolled proteolytic degradation by elastases has been implicated in a number of pathological conditions. Human neutrophil elastase (hNE), a member of the chymotrypsin superfamily of serine proteases is a 33-KDa enzyme stored in the azurophilic granules of the neutrophils. In neutrophils the concentration of NE exceeded 5 mM and its total cellular amount has been estimated to be up to 3 pg. Upon activation, NE is rapidly released from the granules into the extracellular space with some portion remaining bound to neutrophil plasma membrane (See Kawabat et al. 2002, Eur. J. Pharmacol. 451, 1-10). The main intracellular physiological function of NE is degradation of foreign organic molecules phagocytosed by neutrophils, whereas the main target for extracellular elastase is elastin (Janoff and Scherer, 1968, J. Exp. Med. 128, 1137-1155). NE is unique, as compared to other proteases (for example, proteinase 3) in that it has the ability to degrade almost all extracellular matrix and key plasma proteins (See Kawabat et al., 2002, Eur. J. Pharmacol. 451, 1-10). It degrades a wide range of extracellular matrix proteins such as elastin, Type 3 and type 4 collagens, laminin, fibronectin, cytokines, etc. (Ohbayashi, H., 2002, Expert Opin. Investig. Drugs, 11, 965-980). NE is a major common mediator of many pathological changes seen in chronic lung disease including epithelial damage (Stockley, R. A. 1994, Am. J. Resp. Crit. Care Med. 150, 109-113).

The destructive role of NE was solidified almost 40 years ago when Laurell and Eriksson reported an association of chronic airflow obstruction and emphysema with deficiency of serum α₁-antitrypsin (Laurell and Eriksson, 1963, Scand. J. Clin. Invest. 15, 132-140). Subsequently it was determined that α₁-antitrypsin is the most important endogenous inhibitor of human NE. The imbalance between human NE and endogenous antiprotease is believed to cause excess human NE in pulmonary tissues which is considered as a major pathogenic factor in chronic obstructive pulmonary disease (COPD). The excessive human NE shows a prominent destructive profile and actively takes part in destroying the normal pulmonary structures, followed by the irreversible enlargement of the respiratory airspaces, as seen mainly in emphysema. There is an increase in neutrophil recruitment into the lungs which is associated with increased lung elastase burden and emphysema in α₁-proteinase inhibitor-deficient mice (Cavarra et al., 1996, Lab. Invest. 75, 273-280). Individuals with higher levels of the NE-α₁ protease inhibitor complex in bronchoalveolar lavage fluid show significantly accelerated decline in lung functions compared to those with lower levels (Betsuyaku et al. 2000, Respiration, 67, 261-267). Instillation of human NE via the trachea in rats causes lung haemorrhage, neutrophil accumulation during acute phase and emphysematous changes during chronic phase (Karaki et al., 2002, Am. J. Resp. Crit. Care Med., 166, 496-500). Studies have shown that the acute phase of pulmonary emphysema and pulmonary haemorrhage caused by NE in hamsters can be inhibited by pre-treatment with inhibitors of NE (Fujie et al., 1999, Inflamm. Res. 48, 160-167).

Neutrophil-predominant airway inflammation and mucus obstruction of the airways are major pathologic features of COPD, including cystic fibrosis and chronic bronchitis. NE impairs mucin production, leading to mucus obstruction of the airways. NE is reported to increase the expression of major respiratory mucin gene, MUC5AC (Fischer, B. M. & Voynow, 2002, Am. J. Respir. Cell Biol., 26, 447-452). Aerosol administration of NE to guinea pigs produces extensive epithelial damage within 20 minutes of contact (Suzuki et al., 1996, Am. J. Resp. Crit. Care Med., 153, 1405-1411). Furthermore NE reduces the ciliary beat frequency of human respiratory epithelium in vitro (Smallman et al., 1984, Thorax, 39, 663-667) which is consistent with the reduced mucociliary clearance that is seen in COPD patients (Currie et al., 1984, Thorax, 42, 126-130). The instillation of NE into the airways leads to mucus gland hyperplasia in hamsters (Lucey et al., 1985, Am. Resp. Crit. Care Med., 132, 362-366). A role for NE is also implicated in mucus hypersecretion in asthma. In an allergen sensitised guinea pig acute asthma model an inhibitor of NE prevented goblet cell degranulation and mucus hypersecretion (Nadel et al., 1999, Eur. Resp. J., 13, 190-196).

NE has been also shown to play a role in the pathogenesis of pulmonary fibrosis. NE: α₁-protenase inhibitor complex is increased in serum of patients with pulmonary fibrosis, which correlates with the clinical parameters in these patients (Yamanouchi et al., 1998, Eur. Resp. J. 11, 120-125). In a murine model of human pulmonary fibrosis, a NE inhibitor reduced bleomycin-induced pulmonary fibrosis (Taooka et al., 1997, Am. J. Resp. Crit. Care Med., 156, 260-265). Furthermore investigators have shown that NE deficient mice are resistant to bleomycin-induced pulmonary fibrosis (Dunsmore et al., 2001, Chest, 120, 35S-36S). Plasma NE level was found to be elevated in patients who progressed to ARDS implicating the importance of NE in early ARDS disease pathogenesis. (Donnelly et al., 1995, Am. J. Res. Crit. Care Med;, 151, 428-1433). The antiproteases and NE complexed with antiprotease are increased in lung cancer area (Marchandise et al., 1989, Eur. Resp. J. 2, 623-629). Recent studies have shown that polymorphism in the promoter region of the NE gene are associated with lung cancer development (Taniguchi et al., 2002, Clin. Cancer Res., 8, 1115-1120.

Acute lung injury caused by endotoxin in experimental animals is associated with elevated levels of NE (Kawabata, et al., 1999, Am. J. Resp. Crit. Care, 161, 2013-2018). Acute lung inflammation caused by intratracheal injection of lipopolysaccharide in mice has been shown to elevate the NE activity in bronchoalveolar lavage fluid which is significantly inhibited by a NE inhibitor (Fujie et al., 1999, Eur. J. Pharmacol., 374, 117-125; Yasui, et al., 1995, Eur. Resp. J., 8, 1293-1299). NE also plays an important role in the neutrophil-induced increase of pulmonary microvascular permeability observed in a model of acute lung injury caused by tumour necrosis factor α (TNFα) and phorbol myristate acetate (PMA) in isolated perfused rabbit lungs (Miyazaki et al., 1998, Am. J. Respir. Crit. Care Med., 157, 89-94).

A role for NE has also been suggested in monocrotoline-induced pulmonary vascular wall thickening and cardiac hypertrophy (Molteni et al., 1989, Biochemical Pharmacol. 38, 2411-2419). Serine elastase inhibitor reverses the monocrotaline-induced pulmonary hypertension and remodelling in rat pulmonary arteries (Cowan et al., 2000, Nature Medicine, 6, 698-702). Recent studies have shown that serine elastase, that is, NE or vascular elastase are important in cigarette smoke-induced muscularisation of small pulmonary arteries in guinea pigs (Wright et al., 2002, Am. J. Respir. Crit. Care Med., 166, 954-960).

NE plays a key role in experimental cerebral ischemic damage (Shimakura et al., 2000, Brain Research, 858, 55-60), ischemia-reperfusion lung injury (Kishima et al., 1998, Ann. Thorac. Surg. 65, 913-918) and myocardial ischemia in rat heart (Tiefenbacher et al., 1997, Eur. J. Physiol., 433, 563-570). Human NE levels in plasma are significantly increased above normal in inflammatory bowel diseases, for example, Crohn's disease and ulcerative colitis (Adeyemi et al., 1985, Gut, 26, 1306-1311). In addition NE has also been assumed to be involved in the pathogenesis of rheumatoid arthritis (Adeyemi et al., 1986, Rheumatol. Int., 6, 57). The development of collagen induced arthritis in mice is suppressed by a NE inhibitor (Kakimoto et al., 1995, Cellular Immunol. 165, 26-32).

Thus, human NE is known as one of the most destructive serine proteases and has been implicated in a variety of inflammatory diseases. The important endogenous inhibitor of human NE is α₁-antitrypsin. The imbalance between human NE and antiprotease is believed to give rise to an excess of human NE resulting in uncontrolled tissue destruction. The protease/antiprotease balance may be upset by a decreased availability of α₁-antitrypsin either through inactivation by oxidants such as cigarette smoke, or as a result of genetic inability to produce sufficient serum levels. Human NE has been implicated in the promotion or exacerbation of a number of diseases such as pulmonary emphysema, pulmonary fibrosis, adult respiratory distress syndrome (ARDS), ischemia reperfusion injury, rheumatoid arthritis and pulmonary hypertension.

WO 02/053543 discloses pyridone derivatives having affinity for cannabinoid 2-type receptor.

The present invention discloses novel quinoline derivatives that are inhibitors of human neutrophil elastase and homologous serine proteases such as proteinase 3 and pancreatic elastase, and are thereby useful in therapy.

### DISCLOSURE OF THE INVENTION

The present invention provides a compound of formula (I) 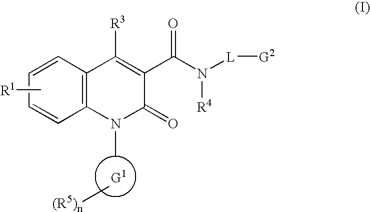
wherein

R¹ represents H, halogen, CN, C1 to 6 alkyl, C1 to 6 alkoxy, CO₂R⁷ or CONR⁸R9;

R³ represents H or F;

G¹ represents phenyl or a five- or six-membered heteroaromatic ring containing 1 to 3 heteroatoms independently selected from O, S and N;

R⁵ represents H, halogen, C1 to 6 alkyl, CN, C1 to 6 alkoxy, NO₂, NR¹⁴R¹⁵, C1 to 3 alkyl substituted by one or more F atoms or C1 to 3 alkoxy substituted by one or more F atoms;

R¹⁴ and R¹⁵ independently represent H or C1 to 3 alkyl; said alkyl being optionally further substituted by one or more F atoms;

n represents an integer 1, 2 or 3 and when n represents 2 or 3, each R⁵ group is selected independently;

R⁴ represents H or C1 to 6 alkyl; said alkyl being optionally further substituted by OH or C1 to 6 alkoxy;

or R⁴ and L are joined together such that the group —NR⁴L represents a 5 to 7 membered azacyclic ring optionally incorporating one further heteroatom selected from O, S and NR¹⁶;

L represents a bond, O, NR²⁹ or C1 to 6 alkyl; said alkyl being optionally incorporating a heteroatom selected from O, S and NR¹⁶; and said alkyl being optionally further substituted by OH or OMe;

G² represents a monocyclic ring system selected from:
i) phenyl or phenoxy,
ii) a 5 or 6 membered heteroaromatic ring containing one to three heteroatoms independently selected from O, S and N,
iii) a C3 to 6 saturated or partially unsaturated cycloalkyl, or
iv) a C4 to 7 saturated or partially unsaturated heterocyclic ring containing one or two heteroatoms independently selected from O, S(O)ₚ and NR¹⁷ and optionally further incorporating a carbonyl group; or

G represents a bicyclic ring system in which each of the two rings is independently selected from:
i) phenyl,
ii) a 5 or 6 membered heteroaromatic ring containing one to three heteroatoms independently selected from O, S and N,
iii) a C3 to 6 saturated or partially unsaturated cycloalkyl, or
iv) a C4 to 7 saturated or partially unsaturated heterocyclic ring containing one or two heteroatoms independently selected from O, S(O)ₚ and NR¹⁷ and optionally further incorporating a carbonyl group;

and the two rings are either fused together, or are bonded directly together or are separated by a linker group selected from O, S(O)_{q} or CH₂,

said monocyclic or bicyclic ring system being optionally further substituted by one to three substituents independently selected from CN, OH, C1 to 6 alkyl, C1 to 6 alkoxy, halogen, NR¹⁸R¹⁹, NO₂, OSO₂R³⁸, CO₂R²⁰, C(═NH)NH₂, C(O)NR²¹R²², C(S)NR^{23 R}²⁴, SC(═NH)NH₂, NR³¹C(═NH)NH₂, S(O)ₛR²⁵, SO₂NR²⁶R²⁷, C1 to 3 alkoxy substituted by one or more F atoms and C1 to 3 alkyl substituted by SO₂R³⁹ or by one or more F atoms; or

when L does not represent a bond, G² may also represent H;

p, q, s and t independently represent an integer 0, 1 or 2;

R⁸ and R⁹ independently represent H or C1 to 6 alkyl; or the group NR⁸R⁹ together represents a 5 to 7 membered azacyclic ring optionally incorporating one further heteroatom selected from O, S and NR²⁸;

R¹⁸ and R¹⁹ independently represent H, C1 to 6 alkyl, formyl, C2 to 6 alkanoyl, S(O)ₜR³² or SO₂NR³³R³⁴; said alkyl group being optionally further substituted by halogen, CN, C1 to 4 alkoxy or CONR⁴¹R⁴²;

R²⁵ represents H, C1 to 6 alkyl or C3 to 6 cycloalkyl; said alkyl group being optionally further substituted by one or more substituents selected independently from OH, CN, CONR³⁵R³⁶, CO₂R³⁷, OCOR⁴⁰, C3 to 6 cycloalkyl, a C4 to 7 saturated heterocyclic ring containing one or two heteroatoms independently selected from O, S(O)ₚ and NR⁴³ and phenyl or a 5 or 6 membered heteroaromatic ring containing one to three heteroatoms independently selected from O, S and N; said aromatic ring being optionally further substituted by one or more substituents selected independently from halogen, CN, C1 to 4 alkyl, C1 to 4 alkoxy, OH, CONR⁴⁴R⁴⁵, CO2R⁴⁶, S(O)ₛR²⁵ or NHCOCH₃;

R³² represents H, C1 to 6 alkyl or C3 to 6 cycloalkyl;

R⁷, R¹⁶, R¹⁷, R²⁰, R²¹, R²², R²³, R²⁴, R²⁶, R²⁷, R²⁸, R²⁹, R³¹, R³³, R³⁴, R³⁵, R³⁶, R³⁷, R³⁸, R³⁹, R⁴⁰, R⁴¹, R⁴², R⁴³, R⁴⁴, R⁴⁵ and R⁴⁶ independently represent H or C1 to 6 alkyl;

and pharmaceutically acceptable salts thereof.

The compounds of formula (I) may exist in enantiomeric and/or tautomeric forms. It is to be understood that all enantiomers, diastereomers, racemates, tautomers and mixtures thereof are included within the scope of the invention.

Unless otherwise indicated, the term “C1 to 6 alkyl” referred to herein denotes a straight or branched chain alkyl group having from 1 to 6 carbon atoms. Examples of such groups include methyl, ethyl, n-propyl, i-propyl, n-butyl, i-butyl, t-butyl, pentyl and hexyl. The terms “C1 to 3 alkyl” and “C1 to 4 alkyl” are to be interpreted analogously.

Examples of “C1 to 3 alkyl substituted by one or more F atoms” include fluoromethyl, difluoromethyl, trifluoromethyl, 2,2,2-trifluoroethyl, 1,1-difluoroethyl, pentafluoroethyl and 3,3,3-trifluoropropyl.

Unless otherwise indicated, the term “C1 to 6 alkoxy ” referred to herein denotes an oxygen substituent bonded to a straight or branched chain alkyl group having from 1 to 6 carbon atoms. Examples of such groups include methoxy, ethoxy, n-propoxy, i-propoxy, n-butoxy, i-butoxy and s-butoxy. The terms “C1 to 3 alkoxy” and “C1 to 4 alkoxy” are to be interpreted analogously.

Examples of “C1 to 3 alkoxy substituted by one or more F atoms” include fluoromethoxy, trifluoromethoxy, 2,2,2-trifluoroethoxy and 3,3,3-trifluoropropoxy.

Unless otherwise indicated, the term “C2 to 6 alkanoyl” referred to herein denotes a straight or branched chain alkyl group having from 1 to 5 carbon atoms bonded to the molecule via a carbonyl group. Examples of such groups include acetyl, propionyl and pivaloyl.

Unless otherwise indicated, the term “halogen” referred to herein denotes fluorine, chlorine, bromine and iodine.

Examples of a five or six membered heteroaromatic ring containing 1 to 3 heteroatoms independently selected from O, S and N include furan, thiophene, pyrrole, oxazole, oxadiazole, isoxazole, imidazole, thiazole, triazole, thiadiazole, pyridine, pyrimidine and pyrazine.

Unless otherwise indicated, the term “C3 to 6 saturated or partially unsaturated cycloalkyl” referred to herein denotes a 3 to 6 membered non-aromatic carbocyclic ring optionally incorporating one or more double bonds. Examples include cyclopropyl, cyclopentyl, cyclopentenyl, cyclohexyl and cyclohexenyl. The term “five- or six-membered saturated or partially unsaturated cycloalkyl ring” is to be interpreted analogously.

Unless otherwise indicated, the term “C4 to 7 saturated or partially unsaturated heterocyclic ring containing one or two heteroatoms independently selected from O, S(O)ₚ and NR¹⁷ and optionally further incorporating a carbonyl group” referred to herein denotes a 4 to 7 membered non-aromatic heterocyclic ring optionally incorporating one or more double bonds and optionally incorporating a carbonyl group. Examples include tetrahydrofuran, thiolane 1,1-dioxide, tetrahydropyran, 4-oxo-4H-pyran, pyrrolidine, pyrroline, imidazolidine, 1,3-dioxolane, piperidine, piperazine, morpholine, perhydroazepine, pyrrolidone and piperidone. The term “five- or six-membered saturated or partially unsaturated heterocyclic ring containing one heteroatom selected from O, S and NR¹³” is to be interpreted analogously.

Examples of a “5 to 7 membered azacyclic ring optionally incorporating one further heteroatom selected from O, S and NR¹⁶” include pyrrolidine, piperidine, morpholine, thiomorpholine and piperazine.

In the definition of L, “C1 to 6 alkyl; said alkyl optionally incorporating a heteroatom selected from O, S and NR¹⁶” embraces a straight or branched chain arrangement of 1 to 6 carbon atoms in which any two carbon atoms are optionally separated by O, S or NR¹⁶. The definition thus includes, for example, methylene, ethylene, propylene, hexamethylene, ethylethylene, —CH₂CH₂O—CH₂—, —CH₂CH₂O—CH₂—CH₂—, —CH₂CH₂S— and —CH₂CH₂NR¹⁶—.

Examples of bicyclic ring systems in which the two rings are either fused together, or are bonded directly together or are separated by a linker group selected from O, S(O)_{q} or CH₂ include biphenyl, thienylphenyl, pyrazolylphenyl, phenoxyphenyl, naphthyl, indanyl, quinolyl, tetrahydroquinolyl, benzofuranyl, indolyl, isoindolyl, indolinyl, benzofuranyl, benzothienyl, indazolyl, benzimidazolyl, benzthiazolyl, purinyl, isoquinolyl, chromanyl, indenyl, quinazolyl, quinoxalyl, chromanyl, isocromanyl, 3H-indolyl, 1H-indazolyl, quinuclidyl, tetrahydronaphthyl, dihydrobenzofuranyl, morpholine-4-ylphenyl, 1,3-benzodioxolyl, 1,1-dioxido-2,3-dihydro-1-benzothienyl, 2,3-dihydro-1,4-benzodioxinyl and 3,4-dihydro-isochromenyl.

In one embodiment, R¹ in formula (I) represents H.

In one embodiment, R³ in formula (I) represents H.

In one embodiment, G¹ in formula (I) represents phenyl or pyridyl. In another embodiment, G¹ in formula (1) represents phenyl.

In one embodiment, R⁵ in formula (I) represents halogen, C1 to 6 alkyl, CN or C1 to 3 alkyl substituted by one or more F atoms. In another embodiment, R⁵ in formula (I) represents Cl, CH₃, CN or CF₃.

In one embodiment, n represents the integer 1.

In another embodiment, G¹ in formula (I) represents phenyl, R⁵ represents CF₃ and n represents the integer 1.

In one embodiment, R⁴ represents H.

In one embodiment, L represents C1 to 6 alkyl. In another embodiment, L represents —CH₂—. In another embodiment, L represents NR²⁹ and R²⁹ represents H.

In one embodiment, G² represents an optionally substituted monocyclic ring system selected from:
i) phenyl,
ii) a 5 or 6 membered heteroaromatic ring containing one to three heteroatoms independently selected from O, S and N,
iii) a C3 to 6 saturated or partially unsaturated cycloalkyl, or p1 iv) a C4 to 7 saturated or partially unsaturated heterocyclic ring containing one or two heteroatoms independently selected from O, S(O)ₚ and NR and optionally further incorporating a carbonyl group.

In another embodiment, G² represents optionally substituted phenyl. In another embodiment, G² represents phenyl substituted by OSO₂R³⁸, S(O)ₛR²⁵, SO₂NR²⁶R²⁷, NR¹⁸R¹⁹ (wherein at least one of R¹⁸ and R¹⁹ represents S(O)ₜR³² or SO2NR³³R³⁴) or C1 to 3 alkyl substituted by SO₂R³⁹.

In another embodiment, G² represents an optionally substituted bicyclic ring system in which each of the two rings is independently selected from:
i) phenyl,
ii) a 5 or 6 membered heteroaromatic ring containing one to three heteroatoms independently selected from O, S and N,
iii) a C3 to 6 saturated or partially unsaturated cycloalkyl, or
iv) a C4 to 7 saturated or partially unsaturated heterocyclic ring containing one or two heteroatoms independently selected from O, S(O)ₚ and NR¹⁷ and optionally further incorporating a carbonyl group;

and the two rings are either fused together, or are bonded directly together or are separated by a linker group selected from O, S(O)_{q} or CH₂.

In one embodiment, R¹ in formula (I) represents H; G¹ represents phenyl; R⁵ represents halogen, C1 to 6 alkyl, CN or C1 to 3 alkyl substituted by one or more F atoms; R⁴ represents H; L represents C1 to 6 alkyl; and G² represents an optionally substituted monocyclic ring system selected from:
i) phenyl,
ii) a 5 or 6 membered heteroaromatic ring containing one to three heteroatoms independently selected from O, S and N,
iii) a C3 to 6 saturated or partially unsaturated cycloalkyl, or
iv) a C4 to 7 saturated or partially unsaturated heterocyclic ring containing one or two heteroatoms independently selected from O, S(O)ₚ and NR¹⁷ and optionally further incorporating a carbonyl group.

In another aspect, the invention specifically provides any compound as described in the Examples herein, or the free base thereof or a pharmaceutically acceptable salt thereof.

Particular compounds include:
N-[4-(methylsulfonyl)benzyl]-2-oxo-1-[3-(trifluoromethyl)phenyl]-1,2-dihydroquinoline-3-carboxamide,
N-(cyclohexylmethyl)-2-oxo-1-[3-(trifluoromethyl)phenyl]-1,2-dihydroquinoline-3-carboxamide;
N-(2-furylmethyl)-2-oxo-1-[3-(trifluoromethyl)phenyl]-1,2-dihydroquinoline-3-carboxamide;
N-[2-(3,4-dimethoxyphenyl)ethyl]-2-oxo-1-[3-(trifluoromethyl)phenyl]-1,2-dihydroquinoline-3-carboxamide;
2-oxo-N-(pyridin-3-ylmethyl)-1-[3-(trifluoromethyl)phenyl]-1,2-dihydroquinoline-3-carboxamide;
N-(3-morpholin-4-ylpropyl)-2-oxo-1-[3-(trifluoromethyl)phenyl]-1,2-dihydroquinoline-3-carboxamide;
2-oxo-N-(1-phenylethyl)-1-[3-(trifluoromethyl)phenyl]-1,2-dihydroquinoline-3-carboxamide;
N-(2-methoxybenzyl)-2-oxo-1-[3-(trifluoromethyl)phenyl]-1,2-dihydroquinoline-3-carboxamide;
N-[2-(4-methoxyphenyl)ethyl]-2-oxo-1-[3-(trifluoromethyl)phenyl)-1,2-dihydroquinoline-3-carboxamide;
2-oxo-N-(2-phenylethyl)-1-[3-(trifluoromethyl)phenyl]-1,2-dihydroquinoline-3-carboxamide;
N-(4-bromobenzyl)-2-oxo-1-[3-(trifluoromethyl)phenyl]-1,2-dihydroquinoline-3-carboxamide;
N-(2-bromobenzyl)-2-oxo-1-[3-(trifluoromethyl)phenyl]-1,2-dihydroquinoline-3-carboxamide;
2-oxo-N-[(2R)-2-phenylcyclopropyl]-1-[3-(trifluoromethyl)phenyl]-1,2-dihydroquinoline-3-carboxamide;
N-[2-(3-chlorophenyl)ethyl]-2-oxo-1-[3-(trifluoromethyl)phenyl]-1,2-dihydroquinoline-3-carboxamide;
N-[(4-cyanocyclohexyl)methyl]-2-oxo-1-[3-(trifluoromethyl)phenyl]-1,2-dihydroquinoline-3-carboxamide;
N-[3-(2-methylpiperidin-1-yl)propyl]-2-oxo-1-[3-(trifluoromethyl)phenyl]-1,2-dihydroquinoline-3-carboxamide;
N-(1-naphthylmethyl)-2-oxo-1-[3-(trifluoromethyl)phenyl]-1,2-dihydroquinoline-3-carboxamide;
N-(3-methylbenzyl)-2-oxo-1-[3-(trifluoromethyl)phenyl]-1,2-dihydroquinoline-3-carboxamide;
N-(2-chlorobenzyl)-2-oxo-1-[3-(trifluoromethyl)phenyl]-1,2-dihydroquinoline-3-carboxamide;
N-(4-methylbenzyl)-2-oxo-1-[3-(trifluoromethyl)phenyl]-1,2-dihydroquinoline-3-carboxamide;
N-(4-fluorobenzyl)-2-oxo-1-[3-(trifluoromethyl)phenyl]-1,2-dihydroquinoline-3-carboxamide;
N-(1,3-benzodioxol-5-ylmethyl)-2-oxo-1-[3-(trifluoromethyl)phenyl]-1,2-dihydroquinoline-3-carboxamide;
N-(2,4-dichlorobenzyl)-2-oxo-1-[3-(trifluoromethyl)phenyl]-1,2-dihydroquinoline-3-carboxamide;
N-(4-chlorobenzyl)-2-oxo-1-[3-(trifluoromethyl)phenyl]-1,2-dihydroquinoline-3-carboxamide;
N-(4-methoxybenzyl)-2-oxo-1-[3-(trifluoromethyl)phenyl]-1,2-dihydroquinoline-3-carboxamide;
N-(3-chlorobenzyl)-2-oxo-1-[3-(trifluoromethyl)phenyl]-1,2-dihydroquinoline-3-carboxamide;
N-(3,4-difluorobenzyl)-2-oxo-1-[3-(trifluoromethyl)phenyl]-1,2-dihydroquinoline-3-carboxamide;
N-(2-chloro-4-fluorobenzyl)-2-oxo-1-[3-(trifluoromethyl)phenyl]-1,2-dihydroquinoline-3-carboxamide;
N-(3,4-dichlorobenzyl)-2-oxo-1-[3-(trifluoromethyl)phenyl]-1,2-dihydroquinoline-3-carboxamide;
N-[2-(2-methoxyphenyl)ethyl]-2-oxo-1-[3-(trifluoromethyl)phenyl]-1,2-dihydroquinoline-3-carboxamide;
N-[2-(4-fluorophenyl)ethyl]-2-oxo-1-[3-(trifluoromethyl)phenyl]-1,2-dihydroquinoline-3-carboxamide;
N-[2-(3-fluorophenyl)ethyl]-2-oxo-1-[3-(trifluoromethyl)phenyl]-1,2-dihydroquinoline-3-carboxamide;
N-[2-(2-fluorophenyl)ethyl]-2-oxo-1-[3-(trifluoromethyl)phenyl]-1,2-dihydroquinoline-3-carboxamide;
N-(2-cyclohex-1-en-1-ylethyl)-2-oxo-1-[3-(trifluoromethyl)phenyl)-1,2-dihydroquinoline-3-carboxamide;
N-(3-methoxybenzyl)-2-oxo-1-[3-(trifluoromethyl)phenyl]-1,2-dihydroquinoline-3-carboxamide;
N-[1-(4-chlorophenyl)ethyl]-2-oxo-1-[3-(trifluoromethyl)phenyl]-1,2-dihydroquinoline-3-carboxamide;
N-(2,5-difluorobenzyl)-2-oxo-1-[3-(trifluoromethyl)phenyl]-1,2-dihydroquinoline-3-carboxamide;
2-oxo-N-[3-(2-oxopyrrolidin-1-yl)propyl]-1-[3-(trifluoromethyl)phenyl]-1,2-dihydroquinoline-3-carboxamide;
2-oxo-N-(pyridin-4-ylmethyl)-1-[3-(trifluoromethyl)phenyl]-1,2-dihydroquinoline-3-carboxamide;
N-(2,3-dihydro-1-benzofuran-5-ylmethyl)-2-oxo-1-[3-(trifluoromethyl)phenyl]-1,2-dihydroquinoline-3-carboxamide;
methyl 4-{[({2-oxo-1-[3-(trifluoromethyl)phenyl]-1,2-dihydroquinolin-3-yl}carbonyl)amino]methyl}benzoate;
2-oxo-N-[2-(2-thienyl)ethyl]-1-[3-(trifluoromethyl)phenyl]-1,2-dihydroquinoline-3-carboxamide;
2-oxo-N-(4-phenoxybenzyl)-1-[3-(trifluoromethyl)phenyl]-1,2-dihydroquinoline-3-carboxamide;
N′-(4-cyanophenyl)-2-oxo-1-[3-(trifluoromethyl)phenyl]-1,2-dihydroquinoline-3-carbohydrazide;
2-oxo-N-(3-thienylmethyl)-1-[3-(trifluoromethyl)phenyl]-1,2-dihydroquinoline-3-carboxamide;
N-[(5-methylisoxazol-3-yl)methyl]-2-oxo-1-[3-(trifluoromethyl)phenyl]-1,2-dihydroquinoline-3-carboxamide;
N-[2-(4-methylphenyl)ethyl]-2-oxo-1-[3-(trifluoromethyl)phenyl]-1,2-dihydroquinoline-3-carboxamide;
N-{2-[4-(aminosulfonyl)phenyl]ethyl}-2-oxo-1-[3-(trifluoromethyl)phenyl]-1,2-dihydroquinoline-3-carboxamide;
2-oxo-N-[4-(1H-pyrazol-1-yl)benzyl]-1-[3-(trifluoromethyl)phenyl]-1,2-dihydroquinoline-3-carboxamide;
2-oxo-N-phenoxy-1-[3-(trifluoromethyl)phenyl]-1,2-dihydroquinoline-3-carboxamide;
N-(2,3-dihydro-1,4-benzodioxin-6-ylmethyl)-2-oxo-1-[3-(trifluoromethyl)phenyl]-1,2-dihydroquinoline-3-carboxamide;
N-(2,3-dihydro-1,4-benzodioxin-2-ylmethyl)-2-oxo-1-[3-(trifluoromethyl)phenyl]-1,2-dihydroquinoline-3-carboxamide;
N-[(6-fluoro-4H-1,3-benzodioxin-8-yl)methyl]-2-oxo-1-[3-(trifluoromethyl)phenyl]-1,2-dihydroquinoline-3-carboxamide;
N-(1-benzothien-3-ylmethyl)-2-oxo-1-[3-(trifluoromethyl)phenyl]-1,2-dihydroquinoline-3-carboxamide;
N-[(4-benzylmorpholin-2-yl)methyl]-2-oxo-1-[3-(trifluoromethyl)phenyl]-1,2-dihydroquinoline-3-carboxamide;
N-[2-(1-methyl-1H-imidazol-4-yl)ethyl]-2-oxo-1-[3-(trifluoromethyl)phenyl]-1,2-dihydroquinoline-3-carboxamide;
N-[2-(1-methyl-1H-imidazol-5-yl)ethyl]-2-oxo-1-[3-(trifluoromethyl)phenyl]-1,2-dihydroquinoline-3-carboxamide;
N-{[3-(4-methoxyphenyl)isoxazol-5-yl]methyl}-2-oxo-1-[3-(trifluoromethyl)phenyl]-1,2-dihydroquinoline-3-carboxamide;
2-oxo-N-[2-(tetrahydro-2H-pyran-4-yl)ethyl]-1-[3-(trifluoromethyl)phenyl]-1,2-dihydroquinoline-3-carboxamide;
N-[3-(3,5-dimethyl-1H-pyrazol-1-yl)propyl]-2-oxo-1-[3-(trifluoromethyl)phenyl]-1,2-dihydroquinoline-3-carboxamide;
N-[(1-methyl-1H-pyrazol-4-yl)methyl]-2-oxo-1-[3-(trifluoromethyl)phenyl]-1,2-dihydroquinoline-3-carboxamide;
2-oxo-N-[(1-phenyl-1H-pyrazol-4-yl)methyl]-1-[3-(trifluoromethyl)phenyl]-1,2-dihydroquinoline-3-carboxamide;
N-{[1-(3-methylphenyl)-1H-pyrazol-4-yl]methyl}-2-oxo-1-[3-(trifluoromethyl)phenyl]-1,2-dihydroquinoline-3-carboxamide;
N-[3-(2-ethylpiperidin-1-yl)propyl]-2-oxo-1-[3-(trifluoromethyl)phenyl]-1,2-dihydroquinoline-3-carboxamide;
N-[(5-methoxy-4-oxo-4H-pyran-2-yl)methyl]-2-oxo-1-[3-(trifluoromethyl)phenyl]-1,2-dihydroquinoline-3-carboxamide;
N-(3-azepan-1-ylpropyl)-2-oxo-1-[3-(trifluoromethyl)phenyl]-1,2-dihydroquinoline-3-carboxamide;
N-[4-(acetylamino)benzyl]-2-oxo-1-[3-(trifluoromethyl)phenyl]-1,2-dihydroquinoline-3-carboxamide;
2-oxo-N-[3-(5-oxo-4,5-dihydro-1H-pyrazol-4-yl)propyl]-1-[3-(trifluoromethyl)phenyl]-1,2-dihydroquinoline-3-carboxamide;
N-[3-(4-methylpiperidin-1-yl)propyl]-2-oxo-1-[3-(trifluoromethyl)phenyl]-1,2-dihydroquinoline-3-carboxamide;
N-[(1,3-dimethyl-1H-pyrazol-4-yl)methyl]-2-oxo-1-[3-(trifluoromethyl)phenyl]-1,2-dihydroquinoline-3-carboxamide;
N-[(1-ethyl-3-methyl-1H-pyrazol-4-yl)methyl]-2-oxo-1-[3-(trifluoromethyl)phenyl]-1,2-dihydroquinoline-3-carboxamide;
N-[(1-ethyl-5-methyl-1H-pyrazol-4-yl)methyl]-2-oxo-1-[3-(trifluoromethyl)phenyl]-1,2-dihydroquinoline-3-carboxamide;
2-oxo-N-(3-piperidin-1-ylpropyl)-1-[3-(trifluoromethyl)phenyl]-1,2-dihydroquinoline-3-carboxamide;
N′-[4-(methylsulfonyl)phenyl]-2-oxo-1-[3-(trifluoromethyl)phenyl]-1,2-dihydroquinoline-3-carbohydrazide;

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids.

In a further aspect the invention provides a process for the preparation of a compound of is formula (I) which comprises reacting a compound of formula (II) 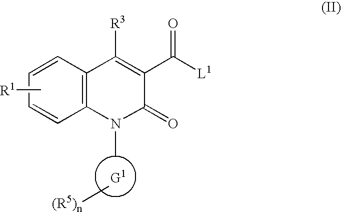
wherein R¹, R³, R⁵, G¹ and n are as defined in formula (I) and L¹ represents a leaving group,

with an amine of formula (III) or a salt thereof 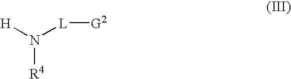
wherein R⁴, G² and L are as defined in formula (I),

and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.

The process is carried out at a suitable temperature, generally between 0° C. and the boiling point of the solvent, in a suitable solvent such as dichloromethane, dioxan or N-methylpyrrolidinone. The process is optionally carried out in the presence of a base and/or a coupling reagent such as HATU, HOAT, HOBT or DIEA. Suitable leaving groups L¹ include OH and halogen, particularly OH or Cl.

Compounds of formula (II) wherein L¹ represents OH may be prepared using methods that will be readily apparent to the man skilled in the art. See, for example, JP 46015097.

Salts of compounds of formula (I) may be formed by reacting the free base or a salt, enantiomer, tautomer or protected derivative thereof, with one or more equivalents of the appropriate acid. The reaction may be carried out in a solvent or medium in which the salt is insoluble, or in a solvent in which the salt is soluble followed by subsequent removal of the solvent in vacuo or by freeze drying. Suitable solvents include, for example, water, dioxane, ethanol, 2-propanol, tetrahydrofuran or diethyl ether, or mixtures thereof. The reaction may be a metathetical process or it may be carried out on an ion exchange resin.

Compounds of formula (I) and intermediate compounds thereto may be prepared as such or in protected form. The protection and deprotection of functional groups is, for example, described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

The compounds of the invention and intermediates may be isolated from their reaction mixtures, and if necessary further purified, by using standard techniques.

The compounds of formula (I) may exist in enantiomeric or diastereoisomeric forms or mixtures thereof, all of which are included within the scope of the invention. The various optical isomers may be isolated by separation of a racemic mixture of the compounds using conventional techniques, for example, fractional crystallisation or HPLC. Alternatively, the individual enantiomers may be made by reaction of the appropriate optically active starting materials under reaction conditions that will not cause racemisation.

Intermediate compounds may also exist in enantiomeric forms and may be used as purified enantiomers, diastereomers, racemates or mixtures thereof.

According to a further aspect of the invention we provide a compound of formula (I) or a pharmaceutically acceptable salt thereof, for use as a medicament.

The compounds of formula (I), and their pharmaceutically acceptable salts, are useful because they possess pharmacological activity in animals. The compounds of formula (I) have activity as pharmaceuticals, in particular as modulators of human neutrophil elastase and homologous serine proteases such as proteinase 3 and pancreatic elastase, and as such are predicted to be useful in therapy. The compounds of formula (I) are particularly useful as inhibitors of human neutrophil elastase. They may thus be used in the treatment or prophylaxis of inflammatory diseases and conditions.

Examples of these conditions are: adult respiratory distress syndrome (ARDS), cystic fibrosis, pulmonary emphysema, chronic obstructive pulmonary disease (COPD) and ischaemic-reperfusion injury. The compounds of this invention may also be useful in the modulation of endogenous and/or exogenous biological irritants which cause and/or propagate atherosclerosis, diabetes, myocardial infarction; hepatic disorders including but not limited to cirrhosis, systemic lupus erythematous, inflammatory disease of lymphoid origin, including but not limited to T lymphocytes, B lymphocytes, thymocytes; autoimmune diseases, bone marrow; inflammation of the joint (especially rheumatoid arthritis, osteoarthritis and gout); inflammation of the gastrointestinal tract (especially inflammatory bowel disease, ulcerative colitis, pancreatitis and gastritis); inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); age related illness such as dementia, inflammatory diseases of cardiovascular origins; granulomatous diseases; renal diseases including but not limited to nephritis and polyarteritis; cancer; pulmonary hypertension, ingested poisons, skin contacts, stings, bites; asthma; rhinitis; HIV disease progression; for minimising the effects of organ rejection in organ transplantation including but not limited to human organs; and replacement therapy of proteinase inhibitors.

Thus, another aspect of the invention provides the use of a compound of formula (I) or a pharmaceutically acceptable salt thereof, in the manufacture of a medicament for the treatment or prophylaxis of diseases or conditions in which inhibition of neutrophil elastase activity is beneficial; and a method of treating, or reducing the risk of, diseases or conditions in which inhibition of neutrophil elastase activity is beneficial which comprises administering to a person suffering from or at risk of, said disease or condition, a therapeutically effective amount of a compound of formula (I) or a pharmaceutically acceptable salt thereof.

In another aspect, the invention provides the use of a compound of formula (I) or a pharmaceutically acceptable salt thereof, in the manufacture of a medicament for the treatment or prophylaxis of inflammatory diseases or conditions; and a method of treating, or reducing the risk of, inflammatory diseases or conditions which comprises administering to a person suffering from or at risk of, said disease or condition, a therapeutically effective amount of a compound of formula (I) or a pharmaceutically acceptable salt thereof.

In particular, the compounds of this invention may be used in the treatment of adult respiratory distress syndrome (ARDS), cystic fibrosis, pulmonary emphysema, chronic obstructive pulmonary disease (COPD), pulmonary hypertension, asthma, rhinitis, ischemia-reperfusion injury, rheumatoid arthritis, osteoarthritis, cancer, atherosclerosis and gastric mucosal injury.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dose of the compound to be administered will depend on the compound employed, the disease being treated, the mode of administration, the age, weight and sex of the patient. Such factors may be determined by the attending physician. However, in general, satisfactory results are obtained when the compounds are administered to a human at a daily dosage of between 0.1 mg/kg to 100 mg/kg (measured as the active ingredient).

The compounds of formula (I) may be used on their own, or in the form of appropriate pharmaceutical formulations comprising the compound of the invention in combination with a pharmaceutically acceptable diluent, adjuvant or carrier. Particularly preferred are compositions not containing material capable of causing an adverse reaction, for example, an allergic reaction. Conventional procedures for the selection and preparation of suitable pharmaceutical formulations are described in, for example, “Pharmaceuticals—The Science of Dosage Form Designs”, M. E. Aulton, Churchill Livingstone, 1988.

According to the invention, there is provided a pharmaceutical formulation comprising preferably less than 95% by weight and more preferably less than 50% by weight of a compound of formula (I) in admixture with a pharmaceutically acceptable diluent or carrier.

We also provide a method of preparation of such pharmaceutical formulations that comprises mixing the ingredients.

The compounds may be administered topically, for example, to the lungs and/or the airways, in the form of solutions, suspensions, HFA aerosols or dry powder formulations, for example, formulations in the inhaler device known as the Turbuhaler®; or systemically, for example, by oral administration in the form of tablets, pills, capsules, syrups, powders or granules; or by parenteral administration, for example, in the form of sterile parenteral solutions or suspensions; or by rectal administration, for example, in the form of suppositories.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound with a carrier substance, for example, a mono-, di- or polysaccharide, a sugar alcohol, or an other polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active compound, with or without a carrier substance, is delivered to the patient.

For oral administration the active compound may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum, titanium dioxide, and the like. Alternatively, the tablet may be coated with a suitable polymer dissolved in a readily volatile organic solvent.

For the preparation of soft gelatine capsules, the compound may be admixed with, for example, a vegetable oil or polyethylene glycol. Hard gelatine capsules may contain granules of the compound using either the above mentioned excipients for tablets. Also liquid or semisolid formulations of the drug may be filled into hard gelatine capsules.

Liquid preparations for oral application may be in the form of syrups or suspensions, for example, solutions containing the compound, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The following Examples are intended to illustrate, but in no way limit the scope of the invention.

General Procedures

¹H NMR spectra were recorded on a Varian Mercury-VX 300 MHz instrument. The central peak of dimethylsulfoxide-d₆ (δ_{H} 2.50 ppm) was used as internal reference. Column chromatography was carried out using silica gel (0.040-0.063 mm, Merck). Unless stated otherwise, starting materials were commercially available. All solvents and commercial reagents were of laboratory grade and were used as received. Unless otherwise stated, organic solutions were dried using anhydrous Na₂SO₄.

LC-MS Conditions : Instrument Agilent 1100; Column: Waters Symmetry 2.1×30 mm; C18 3.5 μm; Mass APCI; Flow rate 0.7 ml/min; Wavelength 254 nm; Solvent A: water +0.1% TFA; solvent B: acetonitrile +0.1% TFA; Gradient 15-95%/B 8 min, 95% B 1 min; retention times (RT) are recorded in minutes.

The following abbreviations are used:

HATU O-(7-Azabenzotriazol-1-yl)-N,N,N′,N′-tetramethyluronium hexafluorophosphate

HOAT 1-Hydroxy-7-azabenzotriazole

DIEA N,N-Diisopropylethylamine

NMP 1-N-Methyl-2-pyrrolidinone

DMF N,N-dimethylformamide

### EXAMPLE 1.1

### N-[4-(Methylsulfonyl)benzyl]-2-oxo-1-[3-(trifluoromethyl)phenyl]-1,2-dihydroquinoline-3-carboxamide

### a) 2-Oxo-1-[-(trifluoromethyl)phenyl]-1,2-dihydroquinolie-3-carboxylic acid

The title compound was prepared by analogy to the procedure disclosed in JP 46015097.

¹H NMR (DMSO-d₆): δ 14.01 (1H, s); 9.09 (1H, s); 8.18 (1H, d); 8.01 (2H, d); 7.93 (1H, t); 7.84 (1H, d); 7.68 (1H, t); 7.47 (1H, t); 6.65 (1H, d).

### b) 2-Oxo-1-[-(trifluoromethyl)phenyl]-1,2-dihydroquinolie-3-carbonyl chloride

To a solution of 2-oxo-1-[-(trifluoromethyl)phenyl]-1,2-dihydroquinolie-3-carboxylic acid (5 g, 15 mmol) in dichloromethane (100 ml), oxalyl chloride (20 ml) and DMF (2 drops) were added. The mixture was stirred at ambient temperature for 15 h. The solvent was evaporated and the crude product was used without purification. Yield 5.3 g.

¹H NMR (CDCl₃): δ 8.90 (1H, s); 7.83 (2H, m); 7.77 (1H, t); 7.57 (2H, m); 7.51 (1H, d); 7.35 (1H, t); 6.64 (1H, d).

### c) N-[4-(Methylsulfonyl)benzyl]-2-oxo-1-[3-(trifluoromethyl)phenyl]-1,2-dihydroquinoline-3-carboxamide

A solution of [4-(methylsulfonyl)benzyl]amine hydrochloride (67 mg, 0.30 mmol) and DIEA (154 μl, 0.90 mmol) in dioxan (2 ml) was added to a solution of 2-oxo-1-[-(trifluoromethyl)phenyl]-1,2-dihydroquinolie-3-carbonyl chloride in dioxan (3 ml). The mixture was stirred for 4 h and then diluted with water (4 ml). This solution was then purified by preparative HPLC to give the title compound (80 mg, 57%).

¹H NMR (DMSO-d₆): δ 9.99 (1H, t); 9.01 (1H, s); 8.10 (1H, d); 8.02-7.75 (6H, m); 7.58 (3H, d); 7.38 (1H, t); 6.57 (1H, d); 4.64 (2H, d); 3.18 (3H, s).

Using the appropriate amine or a salt thereof, the following compounds were prepared by a method analogous to that described in Example 1.1.

### EXAMPLE 2.1

### N-(Cyclohexylmethyl)-2-oxo-1-[3-(trifluoromethyl)phenyl]-1,2-dihydroquinoline-3-carboxamide

LC-MS RT: 6.59 min, m/z 429.2 [MH⁺].

### EXAMPLE 2.2

### N-(2-Furylmethyl)-2-oxo-1-[3-(trifluoromethyl)phenyl]-1,2-dihydroquinoline-3-carboxamide

LC-MS RT: 5.52 min, m/z 413.2 [MH⁺].

### EXAMPLE 2.3

### N-[2-(3,4-Dimethoxyphenyl)ethyl]-2-oxo-1-[3-(trifluoromethyl)phenyl]-1,2-dihydroquinoline-3-carboxamide

LC-MS RT: 5.56 min, m/z 497.2 [MH⁺].

### EXAMPLE 2.4

### 2-Oxo-N-(pyridin-3-ylmethyl)-1-[3-(trifluoromethyl)phenyl]-1,2-dihydroquinoline-3-carboxamide

LC-MS RT: 3.56 min, m/z 424.2 [MH⁺].

### EXAMPLE 2.5

### N-(3-Morpholin-4-ylpropyl)-2-oxo-1-[3-(trifluoromethyl)phenyl]-1,2-dihydroquinoline-3-carboxamide

LC-MS RT: 3.56 min, m/z 460.2 [MH⁺].

### EXAMPLE 2.6

### 2-Oxo-N-(1-phenylethyl)-1-[3-(trifluoromethyl)phenyl]-1,2-dihydroquinoline-3-carboxamide

LC-MS RT: 6.14 min, m/z 437.2 [MH⁺].

### EXAMPLE 2.7

### N-(2-Methoxybenzyl)-2-oxo-1-[3-(trifluoromethyl)phenyl]-1,2-dihydroquinoline-3-carboxamide

LC-MS RT: 5.95 min, m/z 453.2 [MH⁺].

### EXAMPLE 2.8

### N-[2-(4-Methoxyphenyl)ethyl-2-oxo-1-[3-(trifluoromethyl)phenyl]-1,2-dihydroquinoline-3-carboxamide

LC-MS RT: 5.95 min, m/z 467.2 [MH⁺].

### EXAMPLE 2.9

### 2-Oxo-N-(2-phenylethyl)-1-[3-(trifluoromethyl)-phenyl]-1,2-dihydroquinoline-3-carboxamide

LC-MS RT: 6.04 min, m/z 437.2 [MH⁺].

### EXAMPLE 2.10

### N-(4-Bromobenzyl)-2-oxo-1-[3-(trifluoromethyl)phenyl]-1,2-dihydroquinoline-3-carboxamide

LC-MS RT: 6.44 min, m/z 501.1 [MH⁺].

### EXAMPLE 2.11

### N-(2-Bromobenzyl)-2-oxo-1-[3-(trifluoromethyl)phenyl]-1,2-dihydroquinoline-3-carboxamide

LC-MS RT: 6.33 min, m/z 501.1 [MH⁺].

### EXAMPLE 2.12

### 2-Oxo-N-[(2R)-2-phenylcyclopropyl]-1-[3-(trifluoromethyl)phenyl]-1,2-dihydroquinoline-3-carboxamide

LC-MS RT: 6.34 min, m/z 449.2 [MH⁺].

### EXAMPLE 2.13

### N-[2-(3-Chlorophenyl)ethyl]-2-oxo-1-[3-(trifluoromethyl)phenyl]-1,2-dihydroquinoline-3-carboxamide

LC-MS RT: 6.41 min, m/z 471.2 [MH⁺].

### EXAMPLE 2.14

### N-[(4-Cyanocyclohexyl)methyl]-2-oxo-1-[3-(trifluoromethyl)phenyl]-1,2-dihydroquinoline-3-carboxamide

LC-MS RT: 5.50 min m/z 454.2 [MH⁺].

### EXAMPLE 2.15

### N-[3-(2-Methylpiperidin-1-yl)propyl]-2-oxo-1-[3-(trifluoromethyl)phenyl]-1,2-dihydroquinoline-3-carboxamide

LC-MS RT: 3.88 min, m/z 472.3 [MH⁺].

### EXAMPLE 2.16

### N-(1-Naphthylmethyl)-2-oxo-1-[3-(trifluoromethyl)phenyl]-1,2-dihydroquinoline-3-carboxamide

LC-MS RT: 6.43 min, m/z 473.2 [MH⁺].

### EXAMPLE 2.17

### N-(3-Methylbenzyl)-2-oxo-1-[3-(trifluoromethyl)phenyl]-1,2-dihydroquinoline-3-carboxamide

LC-MS RT: 6.20 min, m/z 437.2 [MH⁺].

### EXAMPLE 2.18

### N-(2-Chlorobenzyl)-2-oxo-1-[3-(trifluoromethyl)phenyl]-1,2-dihydroquinoline-3-carboxamide

LC-MS RT: 6.24 min, m/z 457.2 [MH⁺].

### EXAMPLE 2.19

### N-(4-Methylbenzyl)-2-oxo-1-[3-(trifluoromethyl)phenyl]-1,2-dihydroquinoline-3-carboxamide

LC-MS RT: 6.20 min, m/z 437.2 [MH⁺].

### EXAMPLE 2.20

### N-(4-Fluorobenzyl)-2-oxo-1-[3-(trifluoromethyl)phenyl]-1,2-dihydroquinoline-3-carboxamide

LC-MS RT: 5.97 min, m/z 441.2 [MH⁺].

### EXAMPLE 2.21

### N-(1,3-Benzodioxol-5-ylmethyl)-2-oxo-1-[3-(trifluoromethyl)phenyl]-1,2-dihydroquinoline-3-carboxamide

LC-MS RT: 5.74 min, m/z 467.2 [MH⁺].

### EXAMPLE 2.22

### N-(2,4-Dichlorobenzyl)-2-oxo-1-[3-(trifluoromethyl)phenyl]-1,2-dihydroquinoline-3-carboxamide

LC-MS RT: 6.78 min, m/z 491.1 [MH⁺].

### EXAMPLE 2.23

### N-(4-Chlorobenzyl)-2-oxo-1-[3-(trifluoromethyl)phenyl]-1,2-dihydroquinoline-3-carboxamide

LC-MS RT: 6.33 min, m/z 457.1 [MH⁺].

### EXAMPLE 2.24

### N-(4-Methoxybenzyl)-2-oxo-1-[3-(trifluoromethyl)phenyl]-1,2-dihydroquinoline-3-carboxamide

LC-MS RT: 5.80 min, m/z 453.2 [MH⁺].

### EXAMPLE 2.25

### N-(3-Chlorobenzyl)-2-oxo-1-[3-(trifluoromethyl)phenyl]-1,2-dihydroquinoline-3-carboxamide

LC-MS RT: 6.31 min, m/z 457.1 [MH⁺].

### EXAMPLE 2.26

### N-(3,4-Difluorobenzyl)-2-oxo-1-[3-(trifluoromethyl)phenyl]-1,2-dihydroquinoline-3-carboxamide

LC-MS RT: 6.12 min, m/z 459.2 [MH⁺].

### EXAMPLE 2.27

### N-(2-Chloro-4-fluorobenzyl)-2-oxo-1-[3-(trifluoromethyl)phenyl]-1,2-dihydroquinoline-3-carboxamide

LC-MS RT: 6.38 min, m/z 475.2 [MH⁺].

### EXAMPLE 2.28

### N-(3,4-Dichlorobenzyl)-2-oxo-1-[3-(trifluoromethyl)phenyl]-1,2-dihydroquinoline-3-carboxamide

LC-MS RT: 6.70 min, m/z 491.1 [MH⁺].

### EXAMPLE 2.29

### N-[2-(2-Methoxyphenyl)ethyl]-2-oxo-1-[3-(trifluoromethyl)phenyl]-1,2-dihydroquinoline-3-carboxamide

LC-MS RT: 6.13 min, m/z 467.2 [MH⁺].

### EXAMPLE 2.30

### N-[2-(4-Fluorophenyl)ethyl]-2-oxo-1-[3-(trifluoromethyl)phenyl]-1,2-dihydroquinoline-3-carboxamide

LC-MS RT: 6.09 min, m/z 455.2 [MH⁺].

### EXAMPLE 2.31

### N-[2-(3-Fluorophenyl)ethyl]-2-oxo-1-[3-(trifluoromethyl)phenyl]-1,2-dihydroquinoline-3-carboxamide

LC-MS RT: 6.09 min, m/z 455.2 [MH⁺].

### EXAMPLE 2.32

### N-[2-(2-Fluorophenyl)ethyl]-2-oxo-1-[3-(trifluoromethyl)phenyl]-1,2-dihydroquinoline-3-carboxamide

LC-MS RT: 6.08 min, m/z 455.2 [MH⁺].

### EXAMPLE 2.33

### N-(2-Cyclohex-1-en-1-ylethyl)-2-oxo-1-[3-(trifluoromethyl)phenyl]-1,2-dihydroquinoline-3-carboxamide

LC-MS RT: 6.64 min, m/z 441.2 [MH⁺].

### EXAMPLE 2.34

### N-(3-Methoxybenzyl)-2-oxo-1-[3-(trifluoromethyl)phenyl]-1,2-dihydroquinoline-3-carboxamide

LC-MS RT: 5.85 min, m/z 453.2 [MH⁺].

### EXAMPLE 2.35

### N-[1-(4-Chlorophenyl)ethyl]-2-oxo-1-[3-(trifluoromethyl)phenyl]-1,2-dihydroquinoline-3-carboxamide

LC-MS RT: 6.58 min, m/z 471.2 [MH⁺].

### EXAMPLE 2.36

### N-(2,5-Difluorobenzyl)-2-oxo-1-[3-(trifluoromethyl)phenyl]-1,2-dihydroquinoline-3-carboxamide

LC-MS RT: 6.84 min, m/z 459.2 [MH⁺].

### EXAMPLE 2.37

### 2-Oxo-N-[3-(2-oxopyrrolidin-1-yl)propyl]-1-[3-(trifluoromethyl)phenyl]-1,2-dihydroquinoline-3-carboxamide

LC-MS RT: 4.36 min, m/z 458.2 [MH⁺].

### EXAMPLE 2.38

### 2-Oxo-N-(pyridin-4-ylmethyl)-1-[3-(trifluoromethyl)phenyl-1,2-dihydroquinoline-3-carboxamide

LC-MS RT: 3.57 min, m/z 424.2 [MH⁺].

### EXAMPLE 2.39

### N-(2,3-Dihydro-1-benzofuran-5-ylmethyl)-2-oxo-1-[3-(trifluoromethyl)phenyl]-1,2-dihydroquinoline-3-carboxamide

LC-MS RT: 5.78 min, m/z 465.2 [MH⁺].

### EXAMPLE 2.40

### Methyl 4-{[({2-oxo-1-[3-(trifluoromethyl)phenyl]-1,2-dihydroquinolin-3-yl}carbonyl)amino]methyl}benzoate

LC-MS RT: 5.76 min, m/z 481.2 [MH⁺].

### EXAMPLE 2.41

### 2-Oxo-N-[2-(2-thienyl)ethyl]-1-[3-(trifluoromethyl)phenyl]-1,2-dihydroquinoline-3-carboxamide

LC-MS RT: 5.89 min, m/z 443.2 [MH⁺].

### EXAMPLE 2.42

### 2-Oxo-N-(4-phenoxybenzyl)-1-[3-(trifluoromethyl)phenyl]-1,2-dihydroquinoline-3-carboxamide

LC-MS RT: 6.80 min, m/z 515.2 [MH⁺].

### EXAMPLE 2.43

### N′-(4-Cyanophenyl)-2-oxo-1-[3-(trifluoromethyl)phenyl]-1,2-dihydroquinoline-3-carbohydrazide

LC-MS RT: 5.36 min, m/z 449.2 [MH⁺].

### EXAMPLE 2.44

### 2-Oxo-N-(3-thienylmethyl)-1-[3-(trifluoromethyl)phenyl]-1,2-dihydroquinoline-3-carboxamide

LC-MS RT: 5.76 min, m/z 429.1 [MH⁺].

### EXAMPLE 2.45

### N-[(5-Methylisoxazol-3-yl)methyl]-2-oxo-1-[3-(trifluoromethyl)phenyl]-1,2-dihydroquinoline-3-carboxamide

LC-MS RT: 5.18 min, m/z 428.2 [MH⁺].

### EXAMPLE 2.46

### N-[2-(4-Methylphenyl)ethyl]-2-oxo-1-[3-(trifluoromethyl)phenyl]-1,2-dihydroquinoline-3-carboxamide

LC-MS RT: 6.36 min, m/z 451.2 [MH⁺].

### EXAMPLE 2.47

### N-{2-[4-(Aminosulfonyl)phenyl]ethyl}-2-oxo-1-[3-(trifluoromethyl)phenyl]-1,2-dihydroquinoline-3-carboxamide

LC-MS RT: 4.86 min, m/z 516.2 [MH⁺].

### EXAMPLE 2.48

### 2-Oxo-N-[4-(1H-pyrazol-1-yl)benzyl]-1-[3-(trifluoromethyl)phenyl]-1,2-dihydroquinoline-3-carboxamide

LC-MS RT: 5.64 min, m/z 489.2 [MH⁺].

### EXAMPLE 2.49

### 2-Oxo-N-phenoxy-1-[3-(trifluoromethyl)phenyl]-1,2-dihydroquinoline-3-carboxamide

LC-MS RT: 5.79 min, m/z 425.2 [MH⁺].

### EXAMPLE 2.50

### N-(2,3-Dihydro-1,4-benzodioxin-6-ylmethyl)-2-oxo-1-[3-(trifluoromethyl)phenyl]-1,2-dihydroquinoline-3-carboxamide

LC-MS RT: 5.71 min, m/z 481.2 [MH⁺].

### EXAMPLE 2.51

### N-(2,3-Dihydro-1,4-benzodioxin-2-ylmethyl)-2-oxo-1-[3-(trifluoromethyl)phenyl]-1,2-dihydroquinoline-3-carboxamide

LC-MS RT: 6.06 min, m/z 481.3 [MH⁺].

### EXAMPLE 2.52

### N-[(6-Fluoro-4H-1,3-benzodioxin-8-yl)methyl]-2-oxo-1-[3-(trifluoromethyl)phenyl]-1,2-dihydroquinoline-3-carboxamide

LC-MS RT: 5.86 min, m/z 499.2 [MH⁺].

### EXAMPLE 2.53

### N-(1-Benzothien-3-ylmethyl)-2-oxo-1-[3-(trifluoromethyl)phenyl]-1,2-dihydroquinoline-3-carboxamide

LC-MS RT: 6.41 min, m/z 479.2 [MH⁺].

### EXAMPLE 2.54

### N-[(4-Benzylmorpholin-2-yl)methyl]-2-oxo-1-[3-(trifluoromethyl)phenyl]-1,2-dihydroquinoline-3-carboxamide

LC-MS RT: 4.12 min, m/z 522.3 [MH⁺].

### EXAMPLE 2.55

### N-[2-(1-Methyl-1H-imidazol-4-yl)ethyl]-2-oxo-1-[3-(trifluoromethyl)phenyl]-1,2-dihydroquinoline-3-carboxamide

LC-MS RT: 3.57 min, m/z 441.2 [MH⁺].

### EXAMPLE 2.56

### N-[2-(1-Methyl-1H-imidazol-5-yl)ethyl]-2-oxo-1-[3-(trifluoromethyl)phenyl]-1,2-dihydroquinoline-3-carboxamide

LC-MS RT: 3.59 min, m/z 441.2 [MH⁺].

### EXAMPLE 2.57

### N-{[3-(4-Methoxyphenyl)isoxazol-5-yl]methyl}-2-oxo-1-[3-(trifluoromethyl)phenyl]-1,2-dihydroquinoline-3-carboxamide

LC-MS RT: 6.04 min, m/z 520.2 [MH⁺].

### EXAMPLE 2.58

### 2-Oxo-N-[2-(tetrahydro-2H-pyran-4-yl)ethyl]-1-[3-(trifluoromethyl)phenyl]-1,2-dihydroquinoline-3-carboxamide

LC-MS RT: 5.17 min, m/z 445.2 [MH⁺].

### EXAMPLE 2.59

### N-[3-(3,5-Dimethyl-1H-pyrazol-1-yl)propyl]-2-oxo-1-[3-(trifluoromethyl)phenyl]-1,2-dihydroquinoline-3-carboxamide

LC-MS RT: 4.09 min, m/z 469.2 [MH⁺].

### EXAMPLE 2.60

### N-[(1-Methyl-1H-pyrazol-4-yl)methyl]-2-oxo-1-[3-(trifluoromethyl)phenyl]-1,2-dihydroquinoline-3-carboxamide

LC-MS RT: 4.44 min, m/z 427.2 [MH⁺].

### EXAMPLE 2.61

### 2-Oxo-N-[(1-phenyl-1H-pyrazol-4-yl)methyl]-1-[3-(trifluoromethyl)phenyl]-1,2-dihydroquinoline-3-carboxamide

LC-MS RT: 5.76 min, m/z 489.2 [MH⁺].

### EXAMPLE 2.62

### N-{[1-(3-Methylphenyl)-1H-pyrazol-4-yl]methyl}-2-oxo-1-[3-(trifluoromethyl)phenyl]-1,2-dihydroquinoline-3-carboxamide

LC-MS RT: 6.08 min, m/z 503.2 [MH⁺].

### EXAMPLE 2.63

### N-[3-(2-Ethylpiperidin-1-yl)propyl]-2-oxo-1-[3-(trifluoromethyl)phenyl]-1,2-dihydroquinoline-3-carboxamide

LC-MS RT: 4.06 min, m/z 486.3 [MH⁺].

### EXAMPLE 2.64

### N-[(5-Methoxy-4-oxo-4H-pyran-2-yl)methyl]-2-oxo-1-[3-(trifluoromethyl)phenyl]-1,2-dihydroquinoline-3-carboxamide

LC-MS RT: 4.37 min, m/z 471.2 [MH⁺].

### EXAMPLE 2.65

### N-(3-Azepan-1-ylpropyl)-2-oxo-1-[3-(trifluoromethyl)phenyl]-1,2-dihydroquinoline-3-carboxamide

LC-MS RT: 3.94 min, m/z 472.3 [MH⁺].

### EXAMPLE 2.66

### N-[4-(Acetylamino)benzyl]-2-oxo-1-[3-(trifluoromethyl)phenyl]-1,2-dihydroquinoline-3-carboxamide

LC-MS RT: 4.79 min, m/z 480.2 [MH⁺].

### EXAMPLE 2.67

### 2-Oxo-N-[3-(5-oxo-4,5-dihydro-1H-pyrazol-4-yl)propyl]-1-[3-(trifluoromethyl)phenyl]-1,2-dihydroquinoline-3-carboxamide

LC-MS RT: 3.96 min, m/z 457.2 [MH⁺].

### EXAMPLE 2.68

### N-[3-(4-Methylpiperidin-1-yl)propyl]-2-oxo-1-[3-(trifluoromethyl)phenyl]-1,2-dihydroquinoline-3-carboxamide

LC-MS RT: 3.59 min, m/z 472.3 [MH⁺].

### EXAMPLE 2.69

### N-[(1,3-Dimethyl-1H-pyrazol-4-yl)methyl]-2-oxo-1-[3-(trifluoromethyl)phenyl]-1,2-dihydroquinoline-3-carboxamide

LC-MS RT: 4.27 min, m/z 441.2 [MH⁺].

### EXAMPLE 2.70

### N-[(1-Ethyl-3-methyl-1H-pyrazol-4-yl)methyl]-2-oxo-1-[3-(trifluoromethyl)phenyl]-1,2-dihydroquinoline-3-carboxamide

LC-MS RT: 4.46 min, m/z 455.2 [MH⁺].

### EXAMPLE 2.71

### N-[(1-Ethyl-5-methyl-1H-pyrazol-4-yl)methyl]-2-oxo-1-[3-(trifluoromethyl)phenyl]-1,2-dihydroquinoline-3-carboxamide

LC-MS RT: 4.50 min, m/z 455.2 [MH⁺].

### EXAMPLE 2.72

### 2-Oxo-N-(3-piperidin-1-ylpropyl)-1-[3-(trifluoromethyl)phenyl]-1,2-dihydroquinoline-3-carboxamide

LC-MS RT: 3.77 min, m/z 458.3 [MH⁺].

### EXAMPLE 2.73

### N′-[4-(Methylsulfonyl)phenyl]-2-oxo-1-[3-(trifluoromethyl)phenyl]-1,2-dihydroquinoline-3-carbohydrazide

LC-MS RT: 4.81 min, m/z 502.2 [MH⁺].

### Screen

Human Neutrophil Elastase Quenched-FRET Assay

The assay uses Human Neutrophil Elastase (HNE) purified from serum (Calbiochem art. 324681; Ref. Baugh, R. J. et al., 1976, Biochemistry. 15, 836-841). HNE was stored in 50 mM NaOAc, 200 mM NaCl, pH 5.5 with added 30% glycerol at −20° C. The protease substrate used was Elastase Substrate V Fluorogenic, MeOSuc-AAPV-AMC (Calbiochem art. 324740; Ref. Castillo, M. J. et al., 1979, Anal. Biochem. 99, 53-64). The substrate was stored in DMSO at −20° C. The assay additions were as follows: Test compounds and controls were added to black 96-well flat-bottom plates (Greiner 655076), 1 μL in 100% DMSO, followed by 30 μL HNE in assay buffer with 0.01% TritonX-100. The assay buffer constitution was: 100 mM Tris (pH 7.5) and 500 mM NaCl. The enzyme and the compounds were incubated at room temperature for 15 minutes. Then 30 μl substrate in assay buffer was added. The assay was stopped after 30 minutes incubation at room temperature by adding 60 μl stop solution (140 mM acetic acid, 200 mM sodium monochloroacetate, 60 mM sodium acetate, pH 4.3). Fluorescence was measured on a Wallac 1420 Victor 2 instrument at settings: Excitation 380 nm, Emission 460 nm. IC₅₀ values were determined using Xlfit curve fitting using model 205.

When tested in the above screen, the compounds of the Examples gave IC₅₀ values for inhibition of human neutrophil elastase activity of less than 30 μM, indicating that the compounds of the invention are expected to possess useful therapeutic properties. Specimen results are shown in the following Table:

| | Inhibition of Human Neutrophil Elastase |
|---|---|
| Compound | IC₅₀ (nM) |
| Example 2.26 | 500 |
| Example 2.51 | 390 |
| Example 2.66 | 320 |

1. A compound of formula (I) 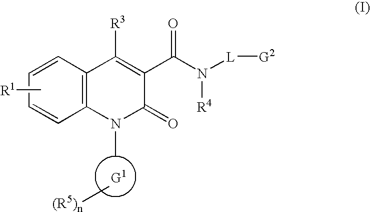
wherein
R¹ represents H, halogen, CN, C1 to 6 alkyl, C1 to 6 alkoxy, CO₂R⁷ or CONR⁸R⁹;
R³ represents H or F;
G¹ represents phenyl or a five- or six-membered heteroaromatic ring containing 1 to 3 heteroatoms independently selected from O, S and N;
R⁵ represents H, halogen, C1 to 6 alkyl, CN, C1 to 6 alkoxy, NO₂, NR¹⁴R¹⁵, C1 to 3 alkyl substituted by one or more F atoms or C1 to 3 alkoxy substituted by one or more F atoms;
R¹⁴ and R¹⁵ independently represent H or C1 to 3 alkyl; said alkyl being optionally further substituted by one or more F atoms;
n represents an integer 1, 2 or 3 and when n represents 2 or 3, each R⁵ group is selected independently;
R⁴ represents H or C1 to 6 alkyl; said alkyl being optionally further substituted by OH or C1 to 6 alkoxy;
or R⁴ and L are joined together such that the group —NR⁴L represents a 5 to 7 membered azacyclic ring optionally incorporating one further heteroatom selected from O, S and NR¹⁶;
L represents a bond, O, NR²⁹ or C1 to 6 alkyl; said alkyl being optionally incorporating a heteroatom selected from O, S and NR¹⁶; and said alkyl being optionally further substituted by OH or OMe;
G² represents a monocyclic ring system selected from:
i) phenyl or phenoxy,
ii) a 5 or 6 membered heteroaromatic ring containing one to three heteroatoms independently selected from O, S and N,
iii) a C3 to 6 saturated or partially unsaturated cycloalkyl, or
iv) a C4 to 7 saturated or partially unsaturated heterocyclic ring containing one or two heteroatoms independently selected from O, S(O)ₚ and NR¹⁷ and optionally further incorporating a carbonyl group; or
G² represents a bicyclic ring system in which each of the two rings is independently selected from:
i) phenyl,
ii) a 5 or 6 membered heteroaromatic ring containing one to three heteroatoms independently selected from O, S and N,
iii) a C3 to 6 saturated or partially unsaturated cycloalkyl, or
iv) a C4 to 7 saturated or partially unsaturated heterocyclic ring containing one or two heteroatoms independently selected from O, S(O)ₚ and NR¹⁷ and optionally further incorporating a carbonyl group;
and the two rings are either fused together, or are bonded directly together or are separated by a linker group selected from O, S(O)_{q} or CH₂,
said monocyclic or bicyclic ring system being optionally further substituted by one to three substituents independently selected from CN, OH, C1 to 6 alkyl, C1 to 6 alkoxy, halogen, NR¹⁸R¹⁹, NO₂, OSO₂R³⁸, CO₂R²⁰, C(═NH)NH₂, C(O)NR²¹R²², C(S)NR²³R²⁴, SC(═NH)NH₂, NR³¹C(═NH)NH₂, S(O)ₛR²⁵, SO₂NR²⁶R²⁷, C1 to 3 alkoxy substituted by one or more F atoms and C1 to 3 alkyl substituted by SO₂R³⁹ or by one or more F atoms; or
when L does not represent a bond, G² may also represent H;
p, q, s and t independently represent an integer 0, 1 or 2;
R⁸ and R⁹ independently represent H or C1 to 6 alkyl; or the group NR⁸R⁹ together represents a 5 to 7 membered azacyclic ring optionally incorporating one further heteroatom selected from O, S and NR²⁸;
R¹⁸ and R¹⁹ independently represent H, C1 to 6 alkyl, formyl, C2 to 6 alkanoyl, S(O)ₜR³ or SO₂NR³³R³⁴; said alkyl group being optionally further substituted by halogen, CN, C1 to 4 alkoxy or CONR⁴¹R⁴²;
R²⁵ represents H, C1 to 6 alkyl or C3 to 6 cycloalkyl; said alkyl group being optionally further substituted by one or more substituents selected independently from OH, CN, CONR³⁵R³⁶; CO₂R³⁷, OCOR⁴⁰, C3 to 6 cycloalkyl, a C4 to 7 saturated heterocyclic ring containing one or two heteroatoms independently selected from O, S(O)ₚ and NR⁴³ and phenyl or a 5 or 6 membered heteroaromatic ring containing one to three heteroatoms independently selected from O, S and N; said aromatic ring being optionally further substituted by one or more substituents selected independently from halogen, CN, C1 to 4 alkyl, C1 to 4 alkoxy, OH, CONR⁴⁴R⁴⁵, CO2R⁴⁶, S(O)ₛR²⁵ or NHCOCH₃;
R represents H, C1 to 6 alkyl or C3 to 6 cycloalkyl;
R⁷, R¹⁶, R¹⁷, R²⁰, R²¹, R²², R²³, R²⁴, R²⁶, R²⁷, R²⁸, R²⁹, R³¹, R³³, R³⁴, R³⁵, R³⁶, R³⁷, R³⁸, R³⁹, R⁴⁰, R⁴¹, R⁴², R⁴³, R⁴⁴, R⁴⁵ and R⁴⁶ independently represent H or C1 to 6 alkyl;
and pharmaceutically acceptable salts thereof.

2. A compound of formula (I), according to claim 1, wherein G¹ represents phenyl.

3. A compound of formula (I), according to claim 1, wherein R⁴ represents H.

4. A compound of formula (I), according to claim 1, wherein R⁵ represents Cl, CH₃, CN or CF₃.

5. (canceled)

6. A pharmaceutical formulation comprising a compound of formula (I), as defined in claim 1 or a pharmaceutically acceptable salt thereof, optionally in admixture with a pharmaceutically acceptable diluent or carrier.

7. A method of treating, or reducing the risk of, a human disease or condition in which inhibition of neutrophil elastase activity is beneficial which comprises administering to a person suffering from or susceptible to such a disease or condition, a therapeutically effective amount of a compound of formula (I), as defined in claim 1, or a pharmaceutically acceptable salt thereof.

8. (canceled)

9. A method of treating or preventing an inflammatory disease or condition, the method comprising administering a therapeutically effective amount of a compound of formula (I) as defined in claim 1, or a pharmaceutically acceptable salt thereof.

10. A process for the preparation of a compound of formula (I), as defined in claim 1, and optical isomers, racemates and tautomers thereof and pharmaceutically acceptable salts thereof, which comprises reacting a compound of formula (II) 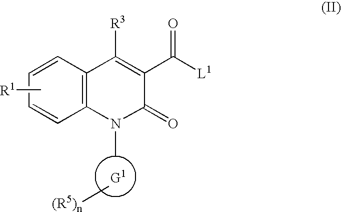
wherein R¹, R³, R⁵, G¹ and n are as defined in claim 1 and L¹ represents a leaving group, with an amine of formula (III) or a salt thereof 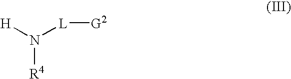
wherein R⁴, G² and L are as defined in claim 1,
and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.
